(19) United States
(12) Patent Application Publication
(10) Pub. No.: US 2006/0115891 A1
(43) Pub. Date: Jun. 1, 2006

(54) Apparatus for minimizing evaporation and/or condensation of samples occurring in tubes of multi-well plate mounted to PCR thermo cycler

(76) Inventor: Gi Young Jang, Seoul (KR)

Correspondence Address: BLAKELY SOKOLOFF TAYLOR & ZAFMAN, 12400 WILSHIRE BOULEVARD, SEVENTH FLOOR, LOS ANGELES, CA, 90025-1030, US

(21) Appl. No.: 11/034,392

(22) Filed: Jan. 11, 2005

(30) Foreign Application Priority Data
Nov. 30, 2004 (KR) 10-2004-0098932

(51) Int. Cl. C12M 1/24

(52) U.S. Cl. 435/288.4; 435/305.4; 435/305.3

(57) ABSTRACT

The present invention relates to an automated apparatus for effectively preventing evaporation of samples in reaction tubes during DNA amplification (i.e., Polymerase Chain Reaction) and DNA sequencing processes performed in a thermo cycler. The automated apparatus of the present invention comprises a plurality of plungers corresponding to the plurality of tubes, respectively, and a coupling member to which the plurality of plungers are simultaneously coupled and fixed. Each of the plurality of plungers includes a rod with a predetermined length, a head which is formed at an upper end of the rod to easily receive an actuating force needed for vertical movement of the plunger into the relevant tube, and an elastic member which is formed around a lower end of the rod to minimize a space defined above a liquid surface of the sample in the tube and is hermetically fitted into the relevant tube. According to the present invention, the evaporation of samples in the reaction tubes occurring during the PCR process can be more effectively prevented and the condensation can also be reduced at the same time, with much simpler configuration and/or operability as compared with the prior art. Therefore, the PCR and DNA sequencing processes can be easily automated.

### BACKGROUND OF THE INVENTION

1. Field of the Invention

The present invention relates to an automated apparatus for effectively preventing evaporation of samples in reaction tubes during DNA amplification and DNA sequencing processes performed in a thermo cycler.

2. Description of the Prior Art

Polymerase Chain Reaction (PCR) is a method of amplifying a specific DNA region to several hundred thousand times by repeating DNA synthesis reactions using DNA polymerases and two kinds of primers with the specific DNA region interposed therebetween, and is a test that is essentially performed in the field of biotechnology.

In general, one cycle of the PCR process comprises the steps of separating double strand DNA into single strand DNA, annealing two kinds of primers with a target region interposed therebetween to be coupled to the separated single strand DNA, and extending the primer to synthesize a complementary sequence to the target region. These steps are preformed at a high temperature while being subjected to a variety of temperatures. Thus, samples in tubes evaporate due the high temperature, and this phenomenon consequently exerts an influence on PCR results. To minimize evaporation of the samples from the tubes, therefore, the following three methods have been generally used.

First, to prevent samples from evaporating from tubes due to the high temperature, mineral oil is added to the sample to allow an oil layer to be formed on the liquid surface of the sample, i.e. an oil layer is deposited onto the liquid surface of the sample. Second, a tube mounted with a lid or cover is used to prevent samples from evaporating from the tube. Finally, the upper open end of the tube is sealed with a sheet made of aluminum or vinyl which is subjected to heat and then formed in conformity with the shape of the upper end of the tube in order to reduce evaporation and/or condensation of the sample in the tube. Alternatively, to prevent the sample from evaporating, a cover made of a special material, which conforms to the shape of the upper end of the tube, may be inserted in the tube.

However, these methods have the following problems. That is, in a case where the liquid surface of the sample is covered with an additional layer of oil, it is required that the oil layer be removed after the PCR process has been completed. The removal of the oil layer is unnecessary and causes the total time period of the PCR process to be increased and an overall process to become more complicated. Further, in a case where a tube mounted with a lid or cover is used or the upper end of the tube is sealed with a sheet made of aluminum or vinyl, an empty space relatively larger than the volume of the sample exists between the cover or sheet and the liquid surface of the sample. Therefore, evaporation of sample corresponding to the volume of the empty space is inevitably produced. Such a degree of evaporation becomes a remarkably large amount of sample in that the amount of sample used in the PCR process is relatively small, it must be avoided. Furthermore, there is a problem in that the lid (or cover) and/or sheet are/is greatly limited in causing the PCR process to be automated.

### SUMMARY OF THE INVENTION

The present invention is conceived to solve the aforementioned problems. In particular, a primary object of the present invention is to provide an apparatus for minimizing unnecessary loss of samples due to the evaporation and/or condensation of samples in tubes or vessels of a multi-well plate mounted to a PCR thermo cycler.

Another object of the present invention is to provide an apparatus for fully automating PCR processes while minimizing the evaporation and/or condensation of samples in the tubes or vessels of the multi-well plate.

To achieve the objects of the present invention, an apparatus of the present invention basically comprises a plurality of structures configured to minimize the evaporation and/or condensation of samples in the respective tubes or vessels of the multi-well plate and a predetermined coupling means for integrally connecting the plurality of structures with one another to automate the PCR processes performed in the multi-well plate.

More specifically, according to an aspect of the present invention, there is provided an apparatus for minimizing evaporation and/or condensation of samples in a plurality of tubes of a multi-well plate mounted to a PCR thermo cycler. The apparatus of the present invention comprises a plurality of plungers corresponding to the plurality of tubes, respectively, and a coupling member to which the plurality of plungers are simultaneously coupled and fixed. Each of the plurality of plungers includes a rod with a predetermined length, a head which is formed at the upper end of the rod to easily receive the actuating force needed for vertical movement of the plunger into the relevant tube, and an elastic member which is formed around the lower end of the rod to minimize the space defined above a liquid surface of the sample in the tube and is hermetically fitted into the relevant tube.

According to another aspect of the present invention, there is provided a plunger for minimizing evaporation and/or condensation of samples in a tube, comprising a rod with a predetermined length and strength; and an O-ring which is formed around a lower end of the rod to minimize a space defined above a liquid surface of the sample in the tube. Further, the O-ring is hermetically fitted into an inner wall of the tube.

According to the present invention, the space above the liquid surface of the sample, i.e. the space where evaporation and/or condensation of a sample may be produced, is minimized. Therefore, the evaporation of samples generally occurring during the PCR and DNA sequencing processes can be more effectively prevented and the condensation of sample can also be reduced. Further, since the coupling member of the present invention is configured in the form of a plate structure to allow the plurality of plungers to be coupled and fixed thereto in such a manner that the plungers are spaced apart from a top surface of the multi-well plate by a predetermined distance, there is an advantage in that the plurality of plungers can be vertically moved more easily and accurately. That is, since 8, 12, 96 384 or 1536 plungers can be coupled onto the flat coupling member at the same time, the plungers can be easily controlled during the PCR or DNA sequencing process, whereby the PCR and DNA sequencing process can also be easily automated.

### BRIEF DESCRIPTION OF THE DRAWINGS

The above and other objects and features of the present invention will become apparent from the following description of preferred embodiments given in connection with the accompanying drawings, in which:

FIG. 1 is a perspective view schematically illustrating a coupling member of the present invention with a plurality of holes, into which plungers are coupled, formed therein;

FIG. 2 is a sectional view schematically illustrating an exemplary plunger and reaction tube according to the present invention;

FIG. 3 is a front view illustrating a state where the plunger shown in FIG. 2 is inserted in and brought into close contact with the reaction tube;

FIG. 4 is a perspective bottom view illustrating an example of a coupling member of the present invention in which a plurality of depressions for coupling with the plungers are further formed;

FIG. 5 is a front view of another plunger according to the present invention;

FIG. 6 is a front view of a further plunger according to the present invention; and

FIG. 7 is a perspective view illustrating an apparatus of the present invention in which a plurality of plungers are coupled with the coupling member.

### DETAILED DESCRIPTION OF THE INVENTION

Hereinafter, preferred embodiments of the present invention will be explained in detail with reference to the accompanying drawings.

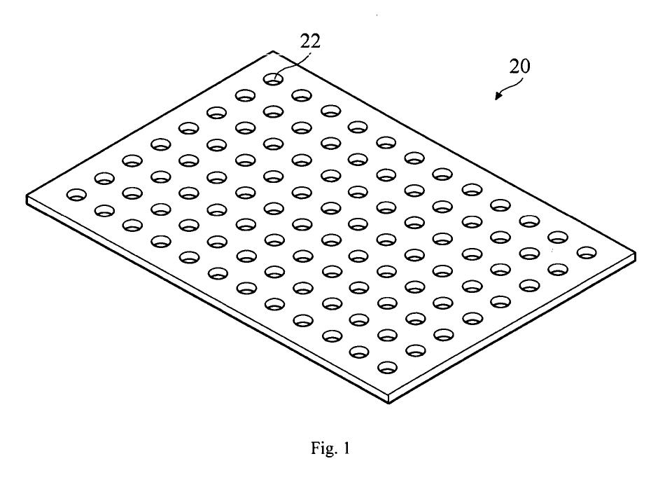

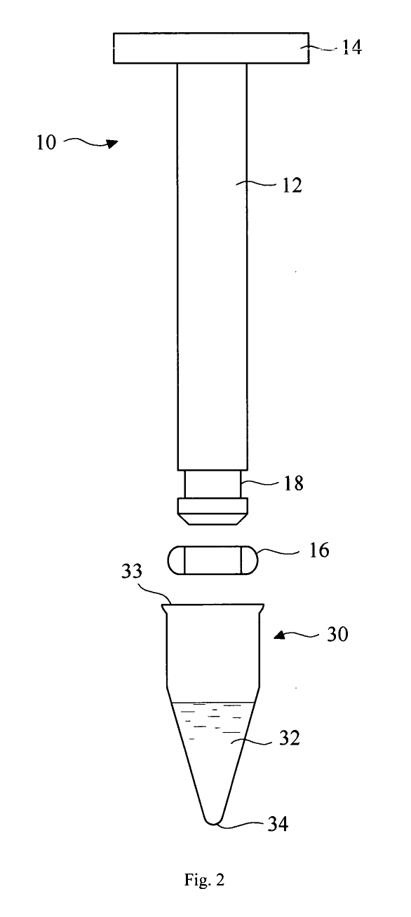

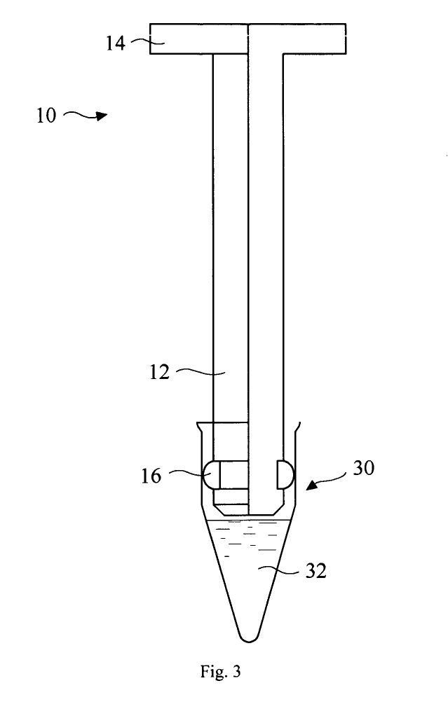

FIG. 1 is a perspective view schematically illustrating a coupling member of the present invention with a plurality of holes, into which plungers are coupled, formed therein; FIG. 2 is a sectional view schematically illustrating an exemplary plunger and reaction tube according to the present invention; and FIG. 3 is a partially cut-away front view iiiustrating a state where the plunger shown in FIG. 2 is inserted in and brought into close contact with the reaction tube.

Referring to FIGS. 1 to 3, an apparatus 1 of the present invention that is used to automate a PCR or DNA sequencing process while minimizing evaporation and/or condensation of samples in reaction tubes or vessels during the process comprises a plurality of plungers 10 corresponding to a plurality of vessels or tubes 30 of a multi-well plate and a coupling member 20 with a predetermined size to which the plurality of plungers 10 are coupled and fixed at the same time.

As well shown in FIG. 2, each of the plurality of plungers 10 includes a vertically extending rod 12, a head 14 which is formed at an upper end of the rod 12 to easily receive an actuating force needed for the vertical motion of the plunger into the relevant reaction vessel or tube 30, and an elastic member 16 which is formed at a lower end of the rod 12 to be inserted into the reaction tube 30 in such a manner that it is brought into close contact with an inner wall of the tube 30.

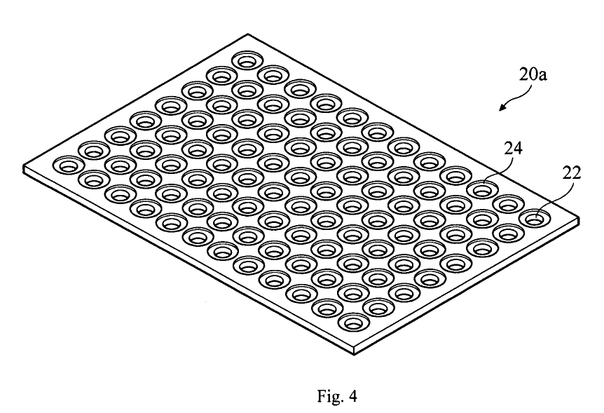

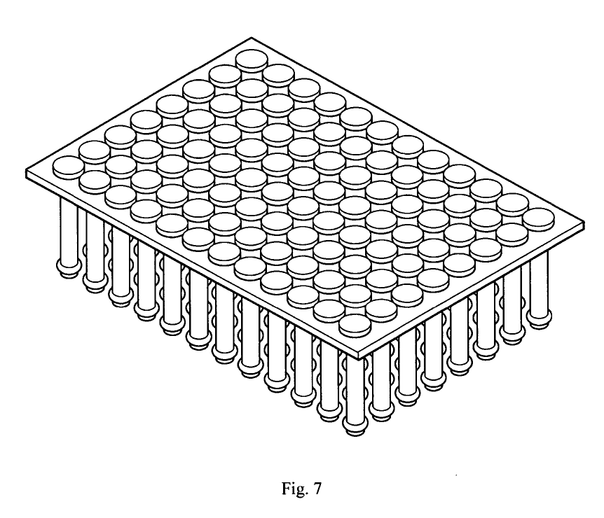

As shown in FIGS. 1 and 4, the plurality of plungers 10 in number suitable to a specific test are coupled and fixed to the coupling member 20 at a time. Such a state is specifically illustrated in FIG. 7. To this end, the coupling member 20 includes holes 22 (refer to FIG. 1) and/or depressions 24 (refer to FIG. 4) in number corresponding to the number of the reaction vessels or tubes of the multi-well plate for use in the PCR process. That is, the holes 22 and/or depressions 24 are formed in the coupling member 20 in number corresponding to the number of the tubes 30 of the multi-well plate used suitably in the PCR thermo cycler. Preferably, the number of the holes 22 or depressions 24 is 96 (=8×12), 364 (=16×24) or 1,536 (=32×48). Each of the rods 22 has the same diameter as that of the rod 12 of the plunger 10. A predetermined portion on a top surface of the coupling member adjacent to the hole 22 and a bottom surface of the head 14 are firmly coupled and fixed to each other using a fixing means such as an adhesive. Further, to increase a coupling force between the plunger and the coupling member, the depression 24 may be employed. Preferably, the depression 24 is formed in the top surface of the coupling member 20 such that it has a predetermined depth (e.g., a half of the thickness of the coupling member) and a size corresponding to the size of the bottom surface of the head 14. Of course, a flour surface of the depression 24 and the bottom surface of the head 14 to be inserted into the depression may be firmly coupled and fixed to each other using such a conventional adhesive as in the previous example.

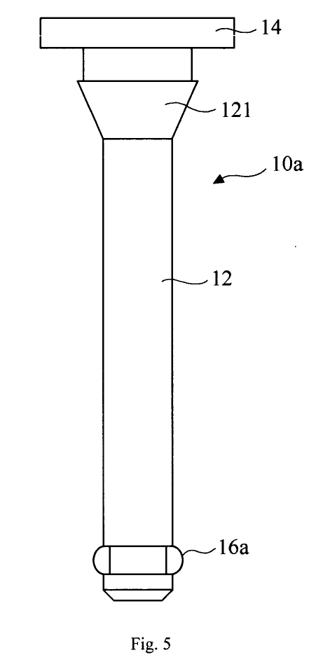

Alternatively, a plunger 10a shown in FIG. 5 may be used. In such a case, no adhesive may be required when the plunger 10a is coupled and fixed to the coupling member 20. That is, the plunger 10a is nearly the same as the plunger 10 shown in FIG. 2 except that it further includes a conical protrusion 121 formed at an upper end of the rod 12. As well shown in FIG. 5, the conical protrusion 121 is slightly expanded upward in a radial direction of the rod 12 from an outer circumferential surface of the rod 12. At this time, an upper end of the conical protrusion 121 and the bottom surface of the head 14 are spaced apart from each other by a predetermined distance that is preferably the same as the thickness t of the coupling member 20. The conical protrusion 121 can be tightly fitted (e.g., “snap-fitted”) into the hole 22 of the coupling member 20 which has the same diameter as that of the rod, because it has suitable elasticity. If the protrusion is tightly fitted into the hole, the movement of the respective plunger 10a is restricted by means of the interaction between a bottom surface of the coupling member 20 adjacent to the hole 22 and a flat surface on the upper end of the conical protrusion 121. Thus, the plunger 10a can be sufficiently coupled and fixed to the coupling member 20 without using a fixing means such as an adhesive as described above. Of course, the adhesive may be used such that the plunger 10a is more firmly coupled and fixed to the coupling member 20.

Furthermore, the coupling member 20 to which the plurality of plungers 10 are coupled may roughly take the shape of a flat plate with holes and/or depressions of which the number is the same as described above. The flat plate should have a predetermined thickness and corresponding stiffness enough to transfer a necessary force needed for pressing the plungers down into the relevant vessels or tubes. Moreover, it is apparent that the flat plate should be strongly fabricated such that the plurality of plungers attached to the coupling member can be moved in a downward vertical direction at the same distance as one another when a force is applied to urge the coupling member toward the reaction vessels or tubes. Similarly, the plungers also have stiffness enough to be properly moved at a predetermined distance by means of a vertical force transmitted from the coupling member. More preferably, the plungers have such characteristics that heat applied to the head can be transferred to the lower end of the rod. The reason is that it is very effective to prevent samples from evaporating from or more particularly condensing in the reaction vessels or tubes in a case where heat whose temperature is similar to the temperature applied to the samples during the PCR process. To this end, it is preferred that the coupling member and plungers be made of a material such as PP, PS, stainless steel and aluminum. Of course, the coupling member and/or plungers may be made of other materials having predetermined stiffness and heat transfer characteristics.

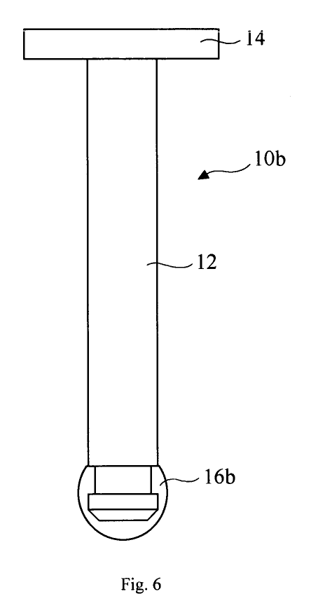

Referring again to FIGS. 2 and 5, in order to bring the elastic member 16 into close contact with the plunger 10 or 10a, an annular recess 18 for use in coupling and fixing the elastic member 16 is preferably formed around an outer circumferential surface of the lower end of the plunger 10 or 10a. Such an annular recess 18 serves to allow the elastic member 16 not to be detached from the plunger 10 or 10a even though the elastic member 16 of the plunger 10 or 10a is repeatedly brought into close contact with and/or retracted from the inner wall of the reaction vessel or tube 30 when the plunger 10 or 10a is inserted into the vessel 30. The elastic member 16 is preferably an O-ring 16a made of a material such as silicone or rubber. Of course, such an O-ring 16a may be replaced with an elastic member 16b in the form of a plug as shown in FIG. 6, so long as it can be ensured that the O-ring is brought into close contact with the inner wall of the reaction vessel 30 when the plunger 16 is inserted into the reaction vessel 30. In such a case, it is preferred that other means similar to the annular recess 18 for use in fixing the O-ring be also used to prevent the plug from be detached from the plunger.

In the meantime, the size (e.g., length) of the plunger 10 or 10a can be determined in accordance with the size (e.g., depth or diameter) of the reaction vessel used. In general, the reaction vessel used in the PCR process is shaped as a tube of which diameter is decreased from its open end 33 to its closed end 34. Preferably, the plunger 10 or 10a has such a length that the bottom surface of the coupling member 20 is spaced apart from the open end 33 by a distance of 0.5 mm to 20 mm when the elastic member 16, such as an O-ring, attached to the lower end of the plunger 10 or 10a is brought into close contact with the inner wall of the relevant reaction vessel 30 at a predetermined position.

According to the present invention, since the coupling member and the plurality of plungers fixedly attached to the coupling member can be configured as described above, the evaporation and condensation of samples in the plurality of reaction vessels or tubes of the multi-well plate can be minimized. That is, since the elastic member in the form of an O-ring is attached to the lower end of the rod of the relevant plunger and brought into close contact with the inner wall of the reaction vessel at a predetermined position, a sample evaporation space in the reaction vessel can be minimized, and thus, the waste of samples can also be reduced. Further, since the plurality of plungers are coupled and fixed to a single coupling member in the form of a flat plate while being spaced apart from the upper end of the reaction vessel by a predetermined distance, the vertical movement of each of the plungers can be easily adjusted. Therefore, the PCR or DNA sequencing process for the plurality of reaction vessels can be easily automated.

Furthermore, since the plurality of plungers are integrally coupled and fixed to the coupling member, a desired amount of heat can be applied to the coupling member and easily transferred then through the relevant plunger to the elastic member positioned adjacent to the sample. Therefore, both condensation and evaporation of samples in the reaction vessel can be prevented.

According to the present invention so configured above, with much simpler configuration and/or operability as compared with methods of using an additional layer such as conventional mineral oil and installing a lid to the upper end of the reaction tube, the evaporation of samples in the reaction tubes occurring during the PCR process can be more effectively prevented and the condensation can also be reduced at the same time. In addition, since the plurality of plungers can be easily mounted to the coupling member in desired number and the vertical movement of the plungers can also be easily controlled, unnecessary processes such as the process of removing oil or attaching/detaching a lid can be beforehand eliminated. Therefore, the PCR and DNA sequencing processes can be easily automated.

While the invention has been shown and described with respect to the preferred embodiments, it will be understood by the skilled in the art that various changes and modifications may be made thereto without departing from the spirit and scope of the invention defined by the appended claims. Accordingly, the present invention should be construed as including the inventions defined by the appended claims and the equivalents thereof.

What is claimed is:

1. An apparatus for minimizing evaporation and/or condensation of samples in a plurality of tubes of a multi-well plate mounted to a PCR thermo cycler, comprising:
a plurality of plungers corresponding to the plurality of tubes, respectively, each of which includes a rod with a predetermined length and an elastic member formed around a lower end of the rod to minimize a space defined above a liquid surface of the sample in the tube, said elastic member being hermetically fitted into the relevant tube, and
a coupling member to which the plurality of plungers are simultaneously coupled and fixed.

2. The apparatus as claimed in claim 1, wherein the elastic member of the plunger is an O-ring made of silicone or rubber.

3. The apparatus as claimed in claim 1, wherein the elastic member of the plunger is a plug made of silicone or rubber.

4. The apparatus as claimed in claim 1, wherein each of the plungers further includes a head formed at an upper end of the rod to easily receive an actuating force needed for vertical movement of the plunger into the relevant tube.

5. The apparatus as claimed in claim 2, wherein each of the plungers further includes a head formed at an upper end of the rod to easily receive an actuating force needed for vertical movement of the plunger into the relevant tube.

6. The apparatus as claimed in claim 4, wherein the head of the plunger is attached to the coupling member at a position corresponding to a position where each of the tubes is formed in the multi-well plate.

7. The apparatus as claimed in claim 4, wherein a plurality of holes are formed in the coupling member and the rod of the plunger is fitted into and fixed to each of the plurality of holes at a position corresponding to a position where each of the tubes is formed in the multi-well plate.

8. The apparatus as claimed in claim 7, wherein a plurality of depressions with predetermined depth, each of which has the same shape as the head, are further formed at portions on a top surface of the coupling member adjacent to the plurality of holes.

9. The apparatus as claimed in claim 8, wherein the head is fixed to the depression with an adhesive.

10. The apparatus as claimed in claim 7, wherein the rod fixed to the hole is formed with a conical protrusion tapering downward at a portion thereof adjacent to the head, and the conical protrusion of which diameter at its uppermost portion is slightly greater than that of the hole is spaced apart downward from the head by a distance corresponding to thickness of the coupling member.

11. The apparatus as claimed in claim 4, wherein the plunger has such a length that a bottom surface of the coupling member is spaced apart from an upper opening of the tube by a distance of 0.5 mm to 20 mm when the elastic member is hermetically fitted into the reaction tube at a predetermined position thereof.

12. The apparatus as claimed in claim 4, wherein the coupling member has stiffness enough to cause the plurality of plungers attached thereto to be kept at the same level as one another.

13. The apparatus as claimed in claim 12, wherein the coupling member is configured to correspond to a shape of an inlet of the multi-well plate of the PCR cycler.

14. The apparatus as claimed in claim 4, wherein the plunger is made of a material capable of transferring heat from the head to the elastic member and withstanding a force applied thereto for vertical movement thereof.

15. The apparatus as claimed in claim 14, wherein the plunger is made of a material selected from a group consisting of PP, PS, stainless steel and aluminum.

16. The apparatus as claimed in claim 1, wherein 8, 12, 96, 384 or 1536 plungers are fixed to the coupling member.

17. The apparatus as claimed in claim 7, wherein 8, 12, 96, 384 or 1536 holes are formed in the coupling member.

18. The apparatus as claimed in claim 8, wherein 8, 12, 96, 384 or 1536 depressions are formed in the coupling member.

19. A plunger for minimizing evaporation and/or condensation of samples in a tube, comprising:
a rod with a predetermined length and strength; and
an O-ring formed around a lower end of the rod to minimize a space defined above a liquid surface of the sample in the tube, said O-ring being hermetically fitted into an inner wall of the tube.

20. The plunger as claimed in claim 19, wherein the O-ring is made of silicone or rubber.

21. The plunger as claimed in claim 19, further comprising a head formed at an upper end of the rod to easily receive an actuating force needed for vertical movement of the plunger into the tube, wherein the head has a sectional area greater than that of the rod.

22. The plunger as claimed in claim 20, further comprising a head formed at an upper end of the rod to easily receive an actuating force needed for vertical movement of the plunger into the tube, wherein the head has a sectional area greater than that of the rod.
